(19) United States
(12) Patent Application Publication
(10) Pub. No.: US 2010/0114167 A1
(43) Pub. Date: May 6, 2010

(54) TRANSITION ROD

(75) Inventors: Bryan S. WILCOX, Collierville, TN (US); Keith E. MILLER, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/262,466

(22) Filed: Oct. 31, 2008

(51) Int. Cl. A61B 17/70; A61B 17/04

(52) U.S. Cl. 606/250; 606/246; 606/301; 606/264

(57) ABSTRACT

A device for supporting vertebral components of a spinal column includes a first spinal rod, a second spinal rod, and a connection element disposed between the first and second rods. The connection element has a front surface with a rod-receiving opening having a first region sized greater than a cross-section of the second rod, and has a second region sized smaller than the cross-section of the second rod, wherein the first and second rods align in series in an end-to-end manner. A locking member is associated with the connection element that urges the second rod toward the second region in a manner that the second rod frictionally engages with the connection element to restrict removal of the second rod from the connection element.

### BACKGROUND OF THE INVENTION

The present invention relates to systems and methods for spinal treatment using spinal rods.

Elongated rigid plates and rods can aid in the stabilization and fixation of a spinal motion segment, in correcting abnormal curvatures and alignments of the spinal column, and for treatment of other conditions. While external rod systems have been employed along the vertebrae, the geometric and dimensional features of these rod systems and patient anatomy constrain the surgeon during surgery and can prevent optimal placement and attachment along the spinal column. For example, elongated, one-piece rods can be difficult to maneuver into position along the spinal column, and also provide the surgeon with only limited options in sizing and selection of the rod system to be placed during surgery.

One known system disclosed in U.S. Publication No. 2005/0277926 to Farris (incorporated herein by reference) includes two rod portions connected together with threaded ends on the rod to provide a modular solution for surgeons. While suitable for many applications, the system can be improved upon to aid in revision surgery and to secure the devices when rods are formed of alternative materials, among other things.

Spinal rod revision surgeries to treat vertebrae adjacent those previously stabilized typically require removal and replacement of the old spinal rod with a new, longer spinal rod. The new rod is typically aligned or bent to provide the same benefits as the old rod. Accordingly, revision surgery requires the same incisions as the original surgery and in some respects, is re-performing a new implantation.

Further, in order to vary rod stiffness, the rod material may be varied at certain spinal levels. Some materials are better suited for a non-threaded solution than others.

A need therefore exists for a spinal rod usable to elongate a previously implanted spinal rod without removing the implanted spinal rod and that may be suitable for securely attaching rods of various materials. The present disclosure overcomes one or more disadvantages of prior spinal rod systems.

### SUMMARY OF THE INVENTION

In one exemplary aspect, the present disclosure is directed to a device for supporting vertebral components of a spinal column. The device includes a first spinal rod, a second spinal rod, and a connection element disposed between the first and second rods. The connection element has a front surface with a rod-receiving opening having a first region sized greater than a cross-section of the second rod, and has a second region sized smaller than the cross-section of the second rod, wherein the first and second rods align in series in an end-to-end manner. A locking member is associated with the connection element that urges the second rod toward the second region in a manner that the second rod frictionally engages with the connection element to restrict removal of the second rod from the connection element.

In some examples, the first spinal rod and the connection element are integrally formed together. In some examples, the first spinal rod is more rigid than the second spinal rod, or the first spinal rod is a metal material and the second spinal rod is a polymeric material. In other examples, the first and second spinal rods have different diameters.

In some examples, the first and second regions form a generally tapering shape, and sides of the taper engage the second rod such that a gap is disposed between the second rod and rod-receiving bore wall directly opposite the locking member.

In another exemplary aspect, the present disclosure is directed to a connection element for receiving and securing a spinal rod. The connection element includes a main body having an upper facing outer surface, a lower facing lower surface, and a front surface. The main body also includes a rod-receiving bore extending inwardly from the front surface to a bore end. The rod-receiving bore has a first region sized greater than a cross-section of the spinal rod, and a second region sized smaller than a cross-section of the spinal rod, such that the first and second regions generally form a tapering shaped opening. The rod receiving bore has a substantially constant inner wall profile extending longitudinally inwardly from the front surface to the bore end. The main body of the connection element also includes a fastener receiving bore formed through the upper facing outer surface in a direction transverse to the direction of the rod-receiving bore. The fastener receiving bore intersects with the rod-receiving bore in an interior region of the connection element. The connection element also includes an integral rod extending rearwardly in a direction opposite the front surface.

In other examples, the connection element includes a rear surface opposite the front surface and includes a second rod-receiving bore extending inwardly from the rear surface in the direction of the front surface to a second bore end. In some examples, the rod-receiving bore is shaped as two overlapping circular bores with the upper circular bore being larger than the lower circular bore.

In yet another exemplary aspect, the present disclosure is directed to a method of implanting a spinal rod. The method includes introducing a first spinal rod in an axial direction to a rod receiving bore formed in a front side of a connection element in a manner that aligns the rod in series with a second spinal rod. The introducing is substantially translational without significant amounts of rotation about a first rod longitudinal axis. A locking element is driven from an adjacent side of the connection element into the rod-receiving bore in a direction transverse to the direction of the rod-receiving bore until the locking element engages the first spinal rod. The first spinal rod is taper-locked in the connection element by using the locking element to force the first spinal rod against at least two surfaces of the rod-receiving bore such that the first spinal rod is engaged between the locking element and the at least two surfaces.

In some examples, the method includes creating an incision and introducing the connection element into the incision, wherein the introducing, driving, and taper locking steps are performed in situ. Accordingly, in some examples, the spinal rod is a previously implanted spinal rod and the surgery is a revision surgery.

These and other advantages of the present invention will be apparent from the descriptions herein.

### BRIEF DESCRIPTION OF THE FIGURES

FIG. 1 is an illustration of a perspective view of first and second assemblies comprising fixation rods attached to vertebral members according to one or more embodiments;

FIG. 2 is an illustration of a perspective view of a device for anchoring a spinal implant in an intervertebral disc space.

FIG. 3 is an illustration of an exploded configuration of the device of FIG. 2.

FIG. 4 is an illustration of an end view of the device of FIG. 2.

FIG. 5 is an illustration of the spinal rod of FIG. 2.

FIG. 6 is an illustration of a top view of the spinal rod of FIG. 2.

FIG. 7 is an illustration of an exploded cross-sectional side view of a portion of the spinal rod in FIG. 2.

FIG. 8 is an illustration of a cross-sectional side view of a portion of the spinal rod in FIG. 2.

FIGS. 9 and 10 are illustrations of alternative embodiments of the spinal rod shown in FIG. 2.

FIGS. 11 and 12 are illustrations of an alternative embodiment showing a connection element that couples to two spinal rods.

### DESCRIPTION OF THE PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present disclosure is directed to a spinal rod for stabilization of one or more vertebra of a spinal column. In some embodiments, the spinal rod includes a transition rod having a connection element connectable to at least one additional spinal rod. The connection element employs a taper-lock system that allows spinal rods of different materials and/or different sizes to be connected in series with one another. Accordingly, while treatment of one vertebral level may require a stiff or rigid rod, an adjacent level may be treated with a rod that is relatively less stiff or rigid. With this connection element, a surgeon may choose any suitable rod size with a suitable material and stiffness and connect it to the transition rod. Therefore, the transition rod addresses both modularity and the attachment of rods of any rigidity. Furthermore, the transition rod curtails adjacent level syndrome and grants surgeons better control over vertebral skipping due to rod design, regardless of surgeon preference. With the transition rod disclosed herein, a surgeon may more easily customize the rod to a patient in the manner desired.

The transition rod's design and structural arrangement also permit a surgeon to effectively elongate a previously implanted spinal rod in a revision surgery. The coupling element on the transition rod is designed to connect to an end of a previously implanted rod, thereby elongating the total rod in situ. The coupling element can receive a rod of any reasonable size because the connection is not dependent on structural features at the end of the rod. Therefore, a surgeon can connect the transition rod to any rod already implanted.

As a further advantage, the transition rod connection elements can connect with a rod irrespective of features at its ends, including rods having smooth outer surfaces. Accordingly, rods made of notch sensitive materials, such as some polymers, may be connected to the transition rod without introducing potentially weakening grooves, notches, threads or other similar characteristics. Accordingly, a broader range of material types are connectable to the transition rod than in conventional systems.

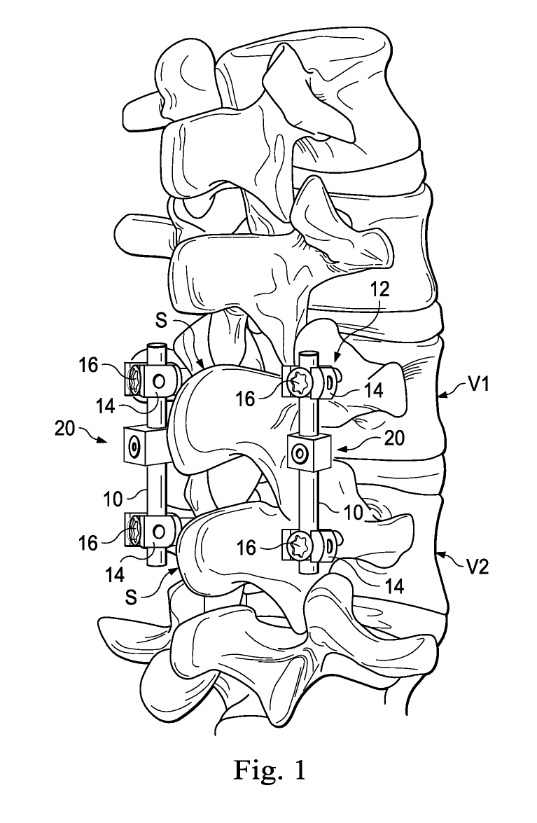

FIG. 1 shows a perspective view of first and second spinal rod assemblies 20 in which spinal rods 10 are attached to vertebral members V1 and V2. In the example assembly 20 shown, the spinal rods 10 are positioned at a posterior side of the spine, on opposite sides of the spinous processes S. Spinal rods 10 may be attached to a spine at other locations, including lateral and anterior locations. The spinal rods 10 may also be attached at various sections of the spine, including the base of the skull and to vertebrae in the cervical, thoracic, lumbar, and sacral regions. Thus, the illustration in FIG. 1 is provided merely as a representative example of one application of a spinal rod 10.

In the exemplary assembly 20, the spinal rods 10 are secured to vertebral members V1, V2 by pedicle assemblies 12 comprising a pedicle screw 14 and a retaining cap 16. The outer surface of the spinal rod 10 is grasped, clamped, or otherwise secured between the pedicle screw 14 and retaining cap 16. In some embodiments, these are multi-axial pedicle screws. Other mechanisms for securing spinal rods 10 to vertebral members V1, V2 include hooks, cables, and other such devices. Further, examples of other types of retaining hardware include threaded caps, screws, and pins. The spinal rods 10 are also attached to plates in other configurations. In some examples, interbody devices or implants, fusion or dynamic, may be disposed between the adjacent vertebrae. Thus, the exemplary assemblies 20 shown in FIG. 1 are merely representative of one type of attachment mechanism.

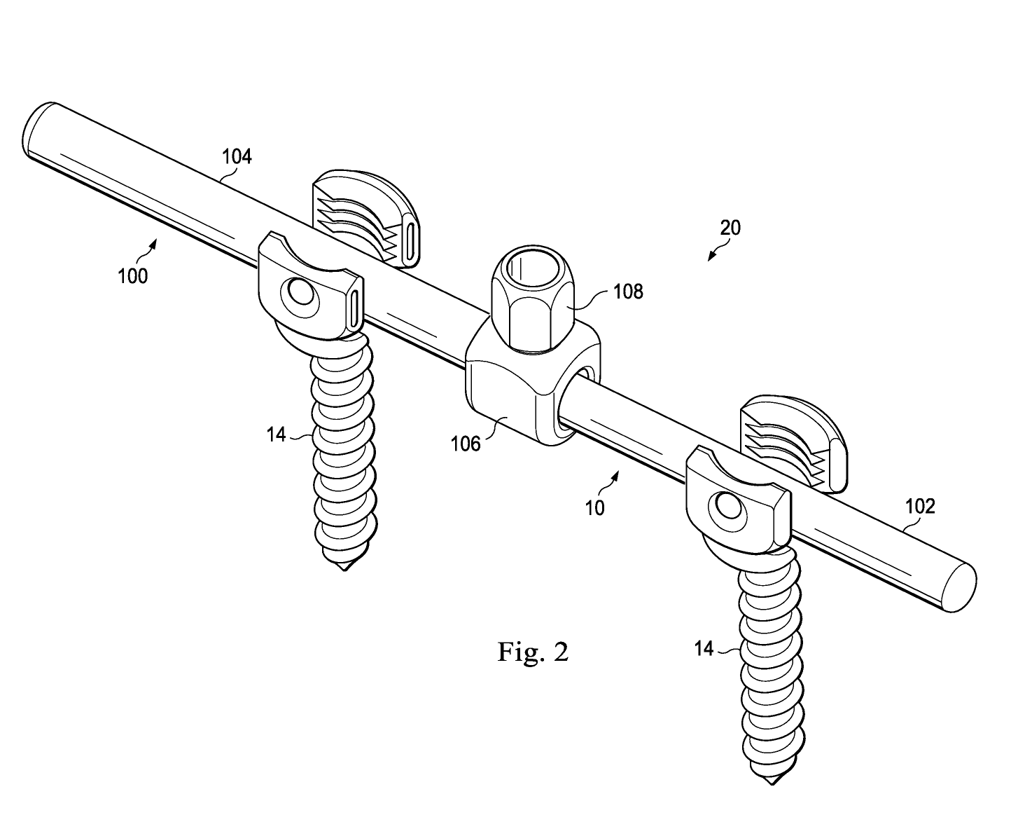

FIG. 2 shows one example of the spinal rod assembly 20 in greater detail. The spinal rod 10 includes a first transition rod 100 and a second rod 102. The transition rod 100 includes a rod portion 104 and a connection element 106 that receives the second rod 102 such that the rod portion 104 and the second rod 102 are releasably coupled to one another in a serial end-to-end fashion. This end-to-end rod coupling arrangement minimizes the footprint or intrusiveness of the coupling mechanism 106 into the tissue surrounding the spinal rod 10, and maximizes the length of the rod portion 104 and the second rod 102 available for positioning and/or attachment along the spinal column. A locking member 108, shown as a set screw, cooperates with the connection element 106 to secure the second rod 102 into the connection element 106.

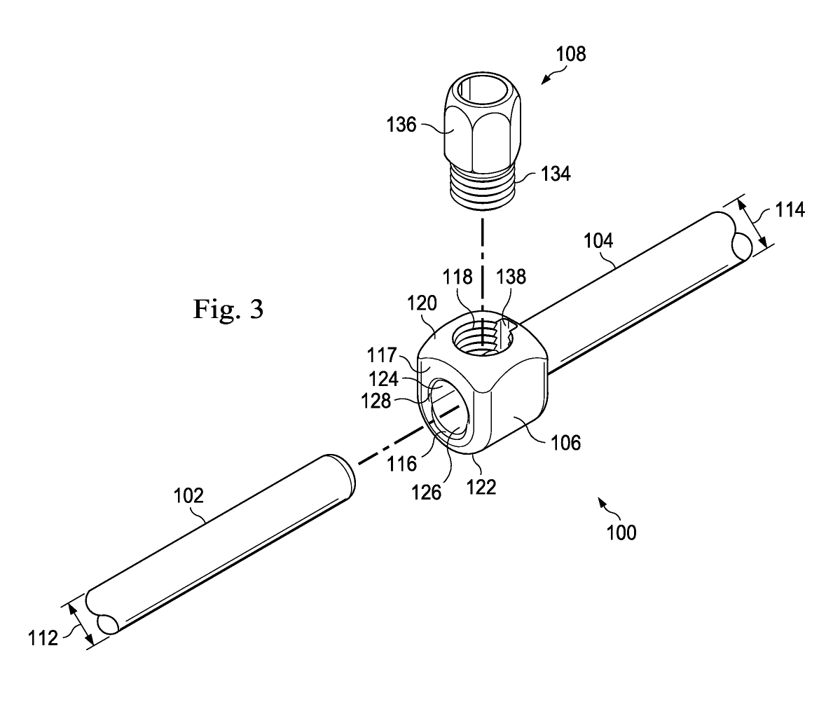

FIG. 3 shows an exploded view of the spinal rod 10. Here, the transition rod 100 includes the rod portion 104 and the connection element 106, with the rod portion 104 extending from the connection element 106. Although not shown in FIGS. 2 or 3, the ends of the first rod portion 104 and the end of the second rod 102 may include another connection element 106 for attachment to additional rods, or may simply terminally end as shown in FIG. 2. Accordingly, in the embodiments illustrated herein, although only one connection element 106 is shown in each rod system 20, one or more of the first and second rods may include an additional connection element or be received into an additional connection element so that three or more rods may comprise the rod system.

An advantage arising from the transition rod 100 disclosed herein is that the first rod portion 104 can be provided with a characteristic that differs from a characteristic of the second rod 102. The connection element 106 allows rods of differing characteristics and rods having the same characteristics to be secured to one another in end-to-end fashion to provide a rod system that is particularly adapted for the anatomy, surgical condition, or surgical procedure. In one embodiment, the characteristic includes a cross-sectional dimension of the rod portions. Other embodiments contemplate selection criteria for selection and assembly of the rod portion to include any one or combination of characteristics, including length, contouring, flexibility, surface features, shape, section modulus, elasticity, materials and material properties, and coatings, for example. For example, in one embodiment a first rod provides a rigid support between a first set of anchors, while the second rod is flexible to provide dynamic stabilization between a second set of anchors. The second rod can be in the form of a tether, cable wire, spring, bumper, or other motion permitting construct.

As indicated above, the second rod 102 may be formed of a material different than the material of the transition rod 100. Either rod may be formed of any of a variety of materials, preferably a biocompatible material. Some examples of materials that can be used include cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Any combination of these materials may also be suitable. For example, a suitable material may include a layer of carbon fiber reinforced PEEK inside an otherwise uniform PEEK material. In one example, the transition rod 100 is formed of a cobalt-chromium material and the second rod 102 is formed of a PEEK material. In another example, both rods are formed of cobalt-chromium material. All suitable combinations are contemplated.

In some embodiments, the second rod 102 is a previously implanted spinal rod and the transition rod 100 is introduced to the previously implanted spinal rod to elongate the rod, or alternatively to replace a certain portion of the previously implanted rod. Accordingly, during a revision surgery, the transition rod 100 may be attached to the implanted rod to provide either additional length, support, or to provide different rod characteristics, such as rigidity.

Referring to FIGS. 3-5, the second rod 102 includes a first cross-sectional dimension 112 between opposite sides thereof and the rod portion 104 includes a second cross-sectional dimension 114 between opposite sides thereof. In some embodiment, these dimensions are similar, while in others, they are different values. In the illustrated embodiment, the cross-sectional dimension 112 corresponds to a diameter of a cylindrical rod portion 102 that is smaller than a diameter corresponding to cross-sectional dimension 114 of the cylindrical rod portion 104. In one specific application, the diameter of second rod portion 102 is sized to extend along a first portion of the spine, such as the cervical region, and the diameter of first rod portion 104 is sized to extend along a second portion of the spine, such as the thoracic region. Other systems contemplate multiple rod portions coupled to one another in end-to-end fashion with characteristics adapted for positioning along any one or combination of the sacral, lumbar, thoracic and cervical regions of the spinal column.

In the example shown, the connection element 106 appears as a flange or hub on the first rod portion 104 that receives, and it couples with the second rod 102 to connect the rod portion 104 and the second rod 102. In the illustrated embodiment, the connection element 106 is generally a rectangular block, although other shapes are also contemplated, such as square, cylindrical, and non-uniform shapes. The connection element 106 includes a rod-receiving first bore 116 formed internally therein that extends inwardly from an end surface 117 of the connection element 106 opposite the rod portion 104. The connection element 106 further includes a fastener receiving second bore 118 extending therein transversely to the rod-receiving first bore 116. As shown in FIG. 3, the second bore 118 can be internally threaded for receipt of the locking member 108. The second bore 118 can also be orthogonal to the first bore 116, although other orientations are also contemplated. For reference, we refer to the surface having the fastener receiving bore 118 as an upper surface 120, and the surface opposite the upper surface 120 as the lower surface 122.

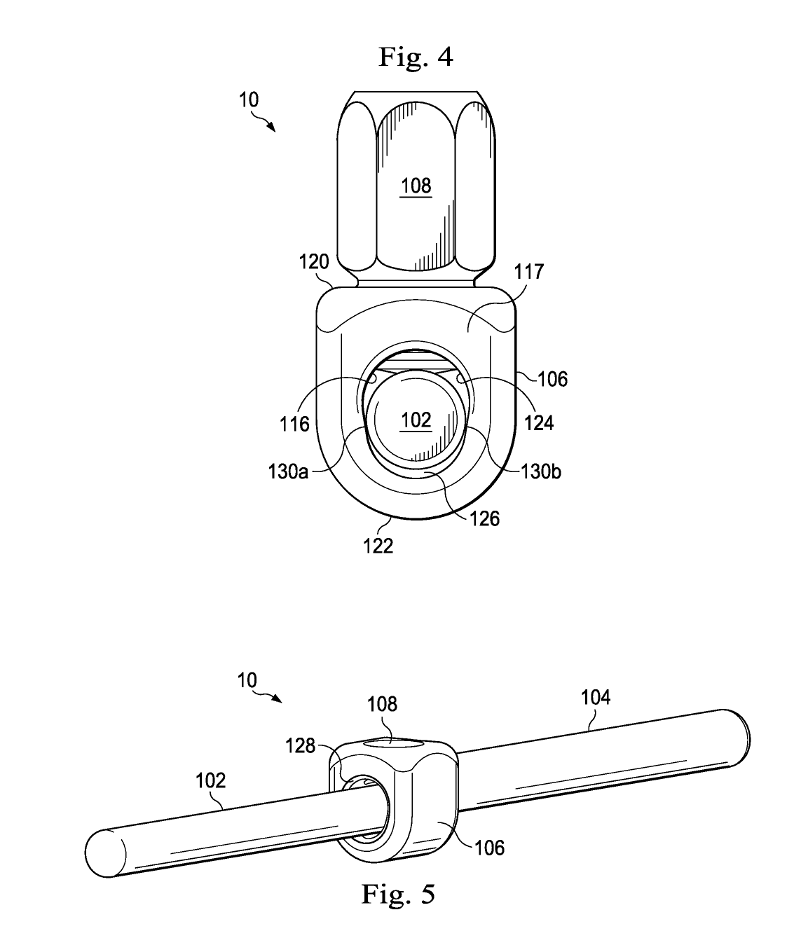

Referring now to FIGS. 3 and 4, the rod-receiving first bore 116 is distinctly shaped to include a greater area at the upper portion and a smaller area the lower portion, such that the width of the rod-receiving bore 116 decreases from the upper portion down, generally forming a tapered bore. Here, the rod-receiving first bore 116 includes a larger bore region 124 and a smaller bore region 126. In the embodiment shown, the larger bore region 124 has an area greater than the smaller bore region and is disposed relatively closer to the upper surface 120 than the smaller bore region 126. The smaller bore region 126 has an area smaller than the larger bore region 124 and is disposed relatively closer to the lower surface 122. The bore regions 124, 126 cooperate to have a width that generally tapers as the distance from the upper surface 120 of the connection element 106 increases. As can be understood with reference to FIG. 4, this greater width at the upper bore region 126 permits a surgeon to easily introduce the second rod 102 into the rod-receiving bore 116, but the narrowing width resulting from the smaller bore region 126 also aids in securing the rod 102 in place in the receiving bore 116 with a taper lock. Accordingly, the upper or larger bore region 124 is sized to be greater than typical conventional spinal rods, while the lower or smaller bore region 126 is sized to be less than typical conventional spinal rods.

In the example shown, the upper bore region 124 and the lower bore region 126 are each formed as overlapping cylindrical bores. The resulting taper creates sides that interface with the second rod 102 at contact points 130a, 130b. The exact location of the contact points 130a, 130b will vary depending on the size of the second rod 102.

In addition, the edge 128 between the inner surface of the bore 116 and the end surface 117 is rounded, chamfered, or both. This not only serves to funnel a rod when its introduced into the connection element 106, but it also protects the rod from nicks or high stress concentrations that may occur if the edge were square. This can be more important when the second rod is formed of polymer materials, such as PEEK, that are notch sensitive. It should be noted that square edges are contemplated for non-notch sensitive materials.

Referring to FIGS. 4 and 5, when a rod is inserted into the larger region of the rod-receiving bore 116, driving the set screw locking member 108 into the connection element 106 against the rod forces the rod 102 downward in the taper until the rod contacts the contact points 130a, 130b. This centralizes the second rod 102 within the rod-receiving bore 116 and, with the locking member 108, taper locks the rod within the connection element 106 with a three-point contact lock. It should be noted that although referred to herein as “three-point,” it is contemplated that the contact need not be a “point” contact in that each contact location may be over an area greater than a single point, and may include an area. One example of this is where the locking member 108 includes a saddle shaped to interface with the rod surface across an area.

In some examples, when the second rod 102 is inserted and secured into the connection element 106, due to the tapering rod-receiving bore 116, the second rod 104 is locked in place and lies above and spaced from the lowermost inner surface portion of the rod receiving bore 116 and below and spaced from the uppermost inner surface of the rod receiving bore 116. Accordingly, gaps are formed both above and below the second rod 102. In the example shown in FIG. 3, the inner surface of the rod-receiving bore 116 is un-threaded, and may include the same inner-wall profile, or a constant inner-wall profile through all or substantially all of the bore axial depth. Accordingly, the contact points 130a, 130b extend all or substantially all the way to the bottom or end of the rod receiving first bore 116. Other arrangements also are contemplated.

Referring now to FIG. 3, the locking member 108 is movably engageable with the connection element 106 to secure the second rod 102 in engagement with the tapering rod-receiving first bore 116. In the example shown, the locking member 108 includes a distal threaded portion 134 and a proximal portion 136. The distal threaded portion 134 is illustrated as an externally threaded set screw that engages the internal thread profile in the screw-receiving second bore 118, although other configurations are contemplated. The proximal portion 136 may include a recess for receive a driving tool, or can be configured to engage a driving tool disposed about its perimeter. In the embodiments shown, the proximal portion 136 can be configured to sever or break-off upon application of a threshold torque. In some embodiments, the distal portion 134 includes an internal bore that can receive a driving tool to facilitate removal or tightening of distal portion 134 after the proximal portion 136 has been broken off or otherwise been removed. FIG. 5 shows the locking member 108 with the proximal portion 136 severed and the rod 102 in place in the connection element 106.

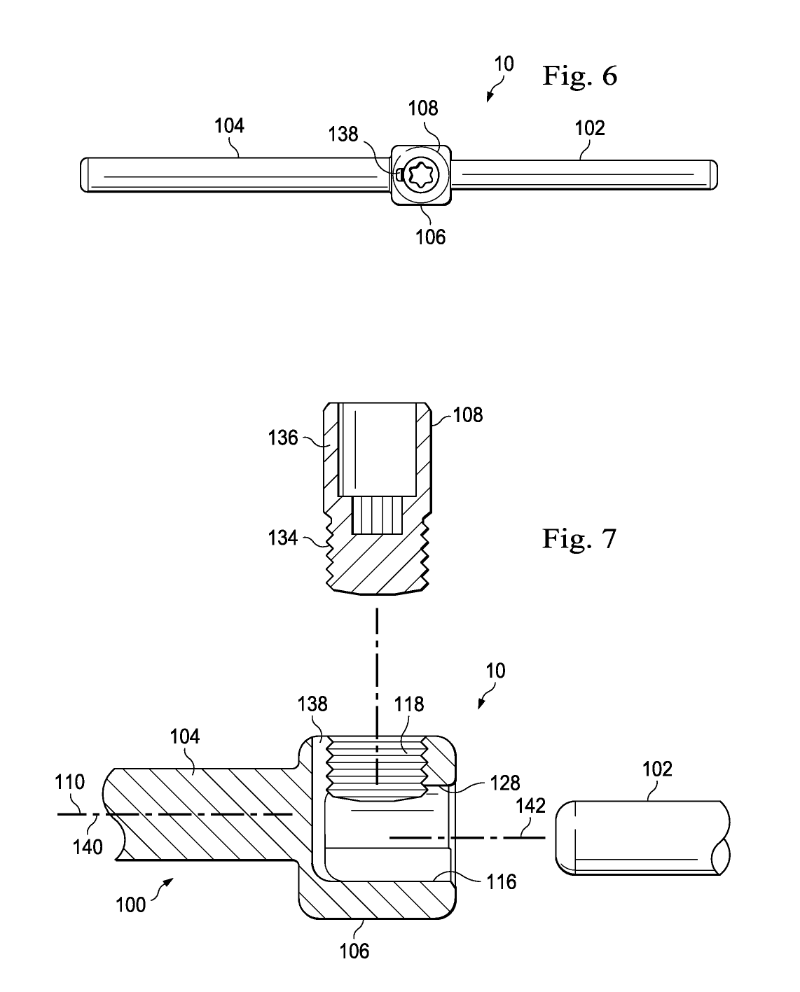

Referring now to FIG. 6, as well as FIG. 3, the screw-receiving second bore 118 includes a view slot 138. This slot enables a surgeon to view the bottom portion of the rod receiving bore 116. In use, through the view slot 138, and with the locking member 108 in place, the surgeon may observe whether the second rod is completely inserted into the rod receiving bore 116. Thus, the surgeon makes a visual determination that the rod is fully seated in the bore 116. Additional ports may be provided in the connection element 106 that permit a surgeon to shine a light into a side of the bore 116 so the surgeon can more easily see whether the second rod is seated.

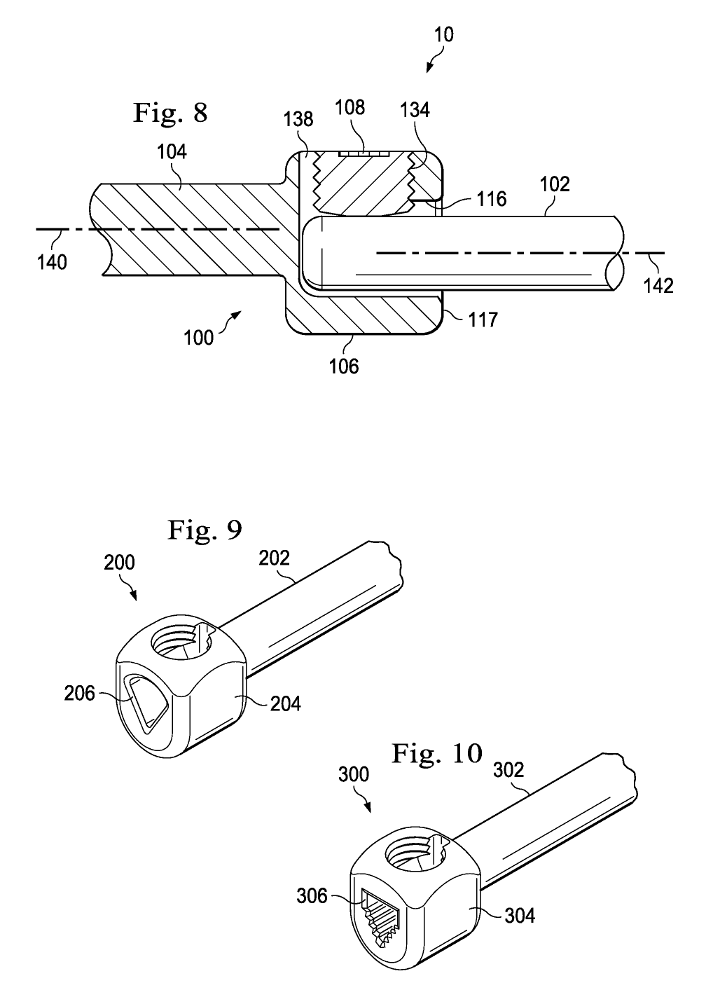

FIGS. 7 and 8 show the spinal rod 10 in cross-section in an exploded configuration and in an assembled configuration, respectively. As can be seen in these and other figures, the spinal rod 10 can be assembled with the transition rod 100 and the second rod 102 in end-to-end fashion. Here, the rod portion 104 includes a first longitudinal axis 140 and the second rod 102 includes a second longitudinal axis 142. The first longitudinal axis 140 may also align with a longitudinal axis or centerline of the rod-receiving bore 116. Because of the tapering cross-section of the rod-receiving bore 116, different rods with different diameters may have respective longitudinal axes that do not align with the longitudinal axis 140 of the rod portion 104. For example, because of the three-point contact, a rod with a relatively smaller diameter sits lower in the tapered rod-receiving first bore 116 than a rod with a relatively larger diameter. Accordingly, in some embodiments, including the example shown in FIG. 8, the axes 140 and 142 are not aligned. In some embodiments, the taper of the rod-receiving first bore is arranged so that when the first rod portion 104 and the second rod 102 have a similar diameter, the axes 140 and 142 align, but when the diameter of the second rod 102 is smaller than the diameter of the rod portion 104, the axes do not align. Accordingly, when the diameter of the second rod 102 is smaller than the diameter of the rod portion 104, the second rod 102 sits lower in the taper, rendering the bottom of the outer surface of the second rod more closely aligned with the bottom surface of the first rod portion. Because the depth of the rods is similar, even when the diameters differ, the spinal rod 10 may be more easily implanted into fasteners, such as screws, that may be driven to the same depth.

In one embodiment, the angle of the taper of the rod-receiving bore 116 is selected so that as the diameter of the second rod decreases, the bottom portion of the second rod substantially aligns with the bottom portion of the rod portion 104.

One advantage of the system disclosed herein is that it is arranged to receive standard cylindrical rod ends, without special treatment or engaging features on the rod ends. Accordingly, it can receive off-the-shelf rods manufactured by any number of manufacturers, including previously implanted rods. Further, because the device employs a taper lock, the device does not rely upon surface features to secure the rod in place. In the example shown, the second rod 102 includes a smooth, cylindrical outer surface. Yet the second rod can securely be locked into the connection element 106. This fixation occurs at least in part because the contact points are not directly opposite each other across the rod. Instead, they are offset from each other, creating the point lock. In addition, tightening the locking member 108 applies high force levels at the contact points due to the point loading. Localizing these force levels at contact points, instead of distributing them throughout the circumference of the smooth rod, creates longitudinal lines of high loading that frictionally resist removal of the rod. In some embodiments, because of the point loading, the rod or the connection element may deform slightly to further frictionally engage and secure the rod in place.

Although the rod is described as smooth and cylindrical, in some embodiments, the rod may have a cross-section other than circular, and may be, for example, oval, rectangular, star-shaped, or other shape. In addition, in some embodiments, the rod end of the inner surface of the rod receiving first bore 116 may be roughened, knurled, peened, or may include other surface imperfections that roughen and increase the frictional engagement of the rod and connection element 116 while still maintaining the rod as substantially smooth.

Still referring to FIG. 8, the round or chamfered edge 128 between the inner surface of the bore 116 and the end surface 118 also assists in distributing the loading applied by the locking member 108. For example, because the point loads are not exactly opposite each other the rod, some rods formed of relatively softer materials, such as polymeric materials, deflect due to the loading applied by the locking member 108. The edge 128 aids in limiting or distributing that deflection by controlling the depth of rod contact within the bore 116.

In use a surgeon may introduce fasteners, such as for example, pedicle screws into vertebrae. The vertebrae may be adjacent or spaced by additional vertebrae. If the surgery is a revision surgery, the spinal rod 10 may be assembled with the previously implanted rod in situ during the process. Likewise, even during a new surgery, the surgeon may choose to assemble the spinal rod 10 in situ.

In a revision surgery, the surgeon may access an end of prior placed spinal rod through an incision. If the revision surgery is to extend the previously placed spinal rod, one or more suitable fasteners, such as the pedicle screws, are inserted into the proper vertebra.

The transition rod 100 may be selected by the surgeon to have difference characteristics than the previously placed rod. It may be formed of a different material, may have a different diameter or surface profile, and/or may have different stiffness. The transition rod 100 may be slid over the end of the previously placed rod so that the previously placed rod is in the connection element 106. Because of the non-circular shape of the rod-receiving bore 116, and because the rod receiving bore has a width that decreases as the distance from the upper surface increases, the rod may be axially slid into the connection element 106 by axial translation, without significant rotation, or any rotation, of the rod about its longitudinal axis. This enables the surgeon to connect the rod, which may be bent to provide desired spinal reinforcement without turning the rod as might occur if the rod were to be threaded into the rod-receiving first bore 116. As indicated above, the relatively smooth, unthreaded interior surface of the connection element permits the rod to also be smooth.

Once the second rod is inserted into the connection element 106, the surgeon may then tighten the locking member 108 to secure the previously implanted rod in place. As the locking member 108 is tightened into the connection element 106, the locking member presses the rod downward toward the region of the bore 116 having a smaller width. Once the rod engages the taper on the connection element 106, any additional tightening increases the frictional forces at the contact points. In the example in FIG. 3, this results in a three-point loading scenario that secures the rod in place in the connection element 106.

When the locking member 108 is a break-off set screw, tightening continues until the break-off portion severs from the threaded distal portion indicating that the proper amount of torque has been achieved. If additional levels are to be connected using additional connection elements, those may be assembled as described above. With the spinal rod assembled, the fasteners may be fully tightened, securing the spinal rod in place. Thus, the rod disclosed herein may be assembled in situ.

When the surgeon assembles the spinal rod 10 prior to implantation, the rod may be assembled as described above, and the rod may be introduced to the fasteners after being fully assembled. Other implantation processes also are contemplated.

FIGS. 9 and 10 show some examples of alternative transition rods, reference herein as 200 and 300, respectively. These transition rods respectively include rod portions 202, 302 and connection elements 204, 304, and each has a rod-receiving bore with a larger first bore region and smaller second bore region. The larger bore region has an area greater than the smaller bore region and is disposed relatively closer to the upper surface than the smaller bore region. The smaller bore region has an area smaller than the larger bore region and is disposed relatively closer to the lower surface. The bore regions cooperate to have a width that generally tapers as the distance from the upper surface of the connection element increases.

The transition rod 200 in FIG. 9 includes a rod-receiving bore 206 that tapers from the top to the bottom, with the bore 206 shaped to be generally triangular, but having an arc forming an upper end that facilitates entry of a second rod. The transition rod 300 in FIG. 10 includes a rod-receiving bore 306 that generally tapers from the top to the bottom, with the bore 306 shaped to be generally triangular, but having stepped surfaces that engage a second rod. These stepped surfaces create point contacts that extend longitudinally along the rod. As with the connection element 106 discussed above, the inner surface features, such as the steps, may extend the length of the bore providing a constant inner wall profile that extends to the end of the bore. It should be noted that these are exemplary only, and other designs also are contemplated. Further, the transition rods 200, 300 may include any feature or benefit discussed with respect to any other embodiment disclosed herein.

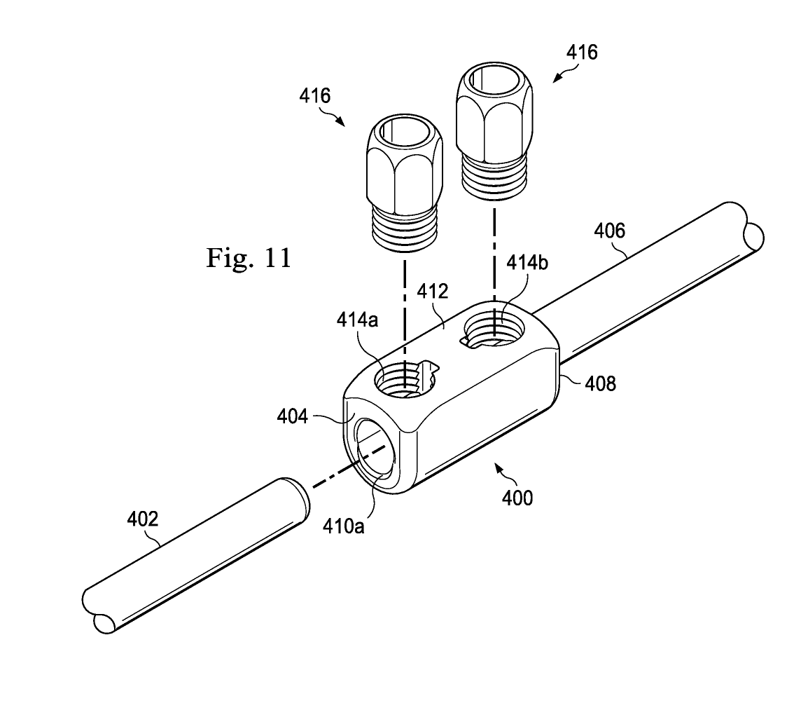

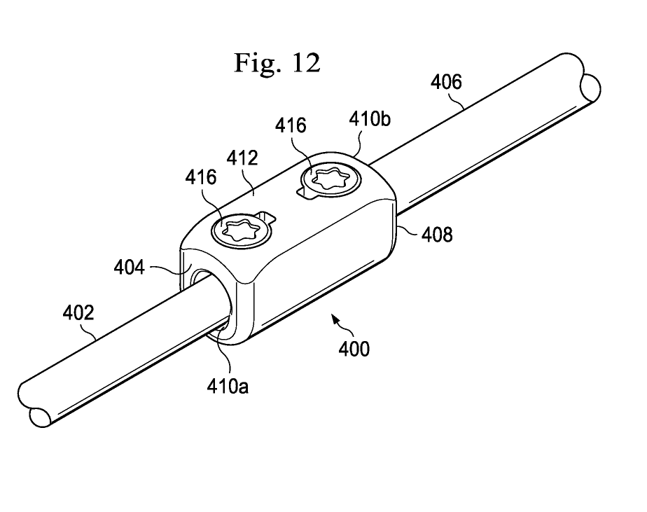

FIGS. 11 and 12 show an alternative embodiment of a connection element, referenced herein by the numeral 400. The connection element 400 is similar to the connection element 106 described above in some respects, but is not integral with a rod portion and instead is configured to receive a separate rod 402 at a first end 404 and receive a separate rod 406 at an opposing second end 408. To this end, each end 404, 408 of the connection element 400 includes rod-receiving bores 410a, 410b, and includes a top surface 412 having two fastener receiving second bores 414a, 414b extending therein transversely to the rod-receiving bores 410. The second bores 414 can be internally threaded for receipt of a locking member 416. Here, the connection element 400 may be considered to be two integral back-to-back connection elements 106 described above.

In this case, the bores 410 do not intersect within the interior of the connection element 400, but each extends inwardly to a bottom wall or bore end (not shown). In other embodiments, the bores 410 intersect each other such that rods inserted in each ends may abut one another within the connection element 400. The connection element 400 may include any feature or benefit discussed with respect to any other embodiment disclosed herein.

The connection element 400 permits a surgeon to select two independent rods for connection via the taper lock. As shown in FIG. 12, the rods 402, 406 are aligned and coupled end-to-end. The rods may include different characteristics as discussed above. Each rod is independently secured using the taper lock formed by the shape of the respective bore 410. Accordingly, longitudinal axes of the rods may be aligned or may be offset in the manner discussed above.

The foregoing detailed description is to be clearly understood as being given by way of illustration and example only, the spirit and scope of the present invention being determined solely by the appended claims.

We claim:

1. A device for supporting vertebral components of a spinal column, comprising:
a first spinal rod;
a second spinal rod;
a connection element disposed between the first and second rods, the connection element having a front surface with a rod-receiving opening having a first region sized greater than a cross-section of the second rod, and a second region sized smaller than the cross-section of the second rod, wherein the first and second rods align in series in an end-to-end manner;
a locking member associated with the connection element that urges the second rod toward the second region in a manner that the second rod frictionally engages with the connection element to restrict removal of the second rod from the connection element.

2. The device of claim 1, wherein the first spinal rod and the connection element are integrally formed together.

3. The device of claim 1, wherein the first spinal rod is more rigid than the second spinal rod.

4. The device of claim 3, wherein the first spinal rod is a metal material and the second spinal rod is a polymeric material.

5. The device of claim 1, wherein the first and second spinal rods have different diameters.

6. The device of claim 1, wherein the first and second regions form a generally tapering shape, and sides of the taper engage the second rod such that a gap is disposed between the second rod and rod-receiving bore wall directly opposite the locking member.

7. The device of claim 1, wherein the second spinal rod includes an end portion having a thread-free, substantially smooth outer perimeter, the end portion being received into the rod-receiving opening.

8. The device of claim 1, wherein the rod-receiving bore and the front surface form an edge, the edge being rounded.

9. The device of claim 1, wherein the connection element includes a viewing slot, at least one of the first and second rods being viewable through the viewing slot to confirm the position of the at least one of the first and second rods within the connection element.

10. A connection element for receiving and securing a spinal rod, comprising:
a main body, comprising:
an upper facing outer surface, a lower facing lower surface, and a front surface;
a rod-receiving bore extending inwardly from the front surface to a bore end, the rod-receiving bore having a first region sized greater than a cross-section of the spinal rod, and a second region sized smaller than a cross-section of the spinal rod, such that the first and second regions generally form a tapering shaped opening, the rod receiving bore having a substantially constant inner wall profile extending longitudinally inwardly from the front surface to the bore end; and
a fastener receiving bore formed through the upper facing outer surface in a direction transverse to the direction of the rod-receiving
bore, the fastener receiving bore intersecting with the rod-receiving
bore in an interior region of the connection element; and
an integral elongate spinal rod extending rearwardly in a direction opposite the front surface of the main body.

11. The connection element of claim 10, comprising:
a rear surface opposite the front surface;
a second rod-receiving bore extending inwardly from the rear surface in the direction of the front surface to a second bore end, the second rod-receiving bore having a first region sized greater than a cross-section of the spinal rod, and a second region sized smaller than a cross-section of the spinal rod, such that the first and second regions generally form a tapering shaped opening, the second rod receiving bore having substantially the same inner wall profile extending longitudinally inwardly from the front surface to the bore end.

12. The connection element of claim 10, wherein the rod-receiving bore is shaped as two overlapping circular bores with the upper circular bore being larger than the lower circular bore.

13. The connection element of claim 10, wherein the rod-receiving bore and the front surface form an edge, the edge being rounded, chamfered, or both.

14. The connection element of claim 10, the fastener receiving bore being threaded to receive a locking member.

15. The connection element of claim 10, including a viewing aperture formed in the main body, the viewing aperture being located and shaped to permit viewing of a rod inserted into the rod-receiving bore to confirm the position of the rod within the rod-receiving bore when the fastener receiving bore includes a fastener.

16. A method of implanting a spinal rod including:
introducing a first spinal rod in an axial direction to a rod receiving bore formed in a front side of a connection element in a manner that aligns the rod in series with a second spinal rod, the introducing being substantially translational without significant amounts of rotation about a first rod longitudinal axis;
driving a locking element from an adjacent side of the connection element into the rod-receiving bore in a direction transverse to the direction of the rod-receiving bore until the locking element engages the first spinal rod; and
taper locking the first spinal rod in the connection element by using the locking element to force the first spinal rod against at least two surfaces of the rod-receiving bore such that the first spinal rod is engaged between the locking element and the at least two surfaces.

17. The method of claim 16, comprising:
creating an incision; and
introducing the connection element into the incision, wherein the introducing, driving, and taper locking steps are performed in situ.

18. The method of claim 17, wherein the spinal rod is a previously implanted spinal rod and the surgery is a revision surgery.

19. The method of claim 16, wherein the connection element includes an integral spinal rod extending therefrom, the method comprising:
driving a fastener into a vertebra;
introducing the integral spinal rod to the fastener; and
securing the integral spinal rod in place at the fastener.

20. The method of claim 16, wherein the spinal rod is a first spinal rod and the connection element connects the first spinal rod to a second spinal rod, the first and second spinal rods having different characteristics.
